(19) United States
(12) Patent Application Publication
(10) Pub. No.: US 2022/0168370 A1
(43) Pub. Date: Jun. 2, 2022

(54) ELASTASE ACTIVITY INHIBITOR, ELASTASE ACTIVITY INHIBITING TOPICAL AGENT, AND ELASTASE ACTIVITY INHIBITING FOOD AND BEVERAGE COMPOSITION

(71) Applicant: MYCOLOGY TECHNO.CORP., Niigata-shi, Niigata (JP)

(72) Inventors: Koji WAKAME, Sapporo-shi, Hokkaido (JP); Yoshiaki TSUNO, Niigata-shi, Niigata (JP)

(21) Appl. No.: 17/600,461

(22) PCT Filed: Mar. 30, 2020
(86) PCT No.: PCT/JP2020/014540

§ 371 Date: Sep. 30, 2021

(30) Foreign Application Priority Data
Apr. 1, 2019 (JP) 2019-069631

(51) Int. Cl. A61K 36/07; A61P 17/14; A61Q 19/08; A61Q 7/00; A23L 33/10; A23L 31/00; A61K 8/9728

(52) U.S. Cl. CPC A61K 36/07; A61P 17/14; A61Q 19/08; A61K 8/9728; A23L 33/10; A23L 31/00; A61Q 7/00

(57) ABSTRACT

A Basidiomycetes-X FERM BP-10011 dry powder or an extract composition thereof is used as an active ingredient.

### TECHNICAL FIELD

The present invention relates to an elastase activity inhibitor, to an elastase activity inhibiting topical agent, and to an elastase activity inhibiting food and beverage composition.

### BACKGROUND ART

Since old days, mushrooms have been frequently used as food materials having unique flavors and tastes. Having physiological function activating actions, such as enhancement of immunocompetence, antimicrobial activity, control of biorhythm, and prevention of senescence, mushrooms have also been used as Chinese herbal medicines or folk medicines for certain types of diseases. Studies of pharmacological ingredients concerned with mushrooms are in progress, resulting in the discovery of ingredients exerting antibacterial and antiviral actions, a cardiotonic action, a hypoglycemic action, a cholesterol-lowering action, an anti-thrombotic action, and an anti-hypertensive action.

The present applicant previously found a novel fungus Basidiomycetes-X FERM BP-10011 (hereinafter referred to simply as “Basidiomycetes-X”) and filed a patent application on an extract composition thereof (hereinafter referred to as a “Basidiomycetes-X extract composition”) (see Patent Document 1). The Basidiomycetes-X extract composition, containing a large amount of polysaccharide (R-D-glucan), exhibits high anti-oxidative power and OH radical-scavenging action. Thus, the composition is expected to exhibit an anti-aging action and the like. The Basidiomycetes-X extract composition, also having an immunomodulating action, is suitably used as an immunoactivator or the like. Separately, the present applicant previously filed a patent application on a composition for ameliorating/preventing an atopic disease, which composition is based on the Basidiomycetes-X extract composition (see Patent Document 2).

Meanwhile, elastin is found in the bodies of mammals (including humans), fishes, etc. The elastic fiber is a type of a fiber belonging to the connective tissue included in the extracellular matrix widely spreading throughout the organs and tissues (e.g., blood vessels, ligaments, lungs, and skin) present in substantially the whole body. The elastic fiber is formed via cross-linking of stretchable a-helix structural units, and is known to play an important role in maintaining flexibility of the tissue. According to a study in skin science, when production of elastic fiber or the like is impeded by reduced proliferation of fibroblasts due to aging, skin aging (i.e., wrinkling, dullness, loss of fineness, and reduction in springiness) occurs. According to another study, when elastic fiber is degraded through external stimuli to the skin including exposure to UV rays, exposure to highly dry air, and excessive cleansing, skin troubles (e.g., roughening and aging) due to wrinkling, dullness, loss of fineness, reduction in springiness, and the like may occur.

One conceivable mechanism of degradation of elastic fiber is decomposition by the mediation of an elastase. Elastase is an enzyme belonging to the serine protease family. Among such proteases, pancreatic elastase increases in the presence of a lesion in the pancreas, and the blood level thereof serves as a marker for a pancreatic disease. The blood level of the elastase originating from leucocytes is known to rise in the presence of microinflammation in an initial stage which cannot be visually confirmed. In the case where the skin is exposed to UV-A, microinflammation is conceivably accumulated in the skin. In this case, an increase in elastase activity is actually confirmed.

Thus, suppression of elastase activity and prevention of degradation of elastic fiber in the dermis are thought to be effective for antiaging of the skin (e.g., wrinkling or sagging). Actually, there have been proposed elastase activity inhibitors each containing, as an active ingredient, a sweet pea extract, an Iceland moss extract, a Ganoderma lucidum extract, glucosamine, a glucosamine derivative, a salt thereof, etc. (see, for example, Patent Documents 3, 4, 5, and 6).

It is also reported that a rise in elastase activity in the skin tissue in response to hair cycle is essential for formation and growth of hair follicles (see, for example, Patent Document 5), and a hair growth inhibitor containing an elastase inhibitor as an active ingredient has been proposed (see, for example, Patent Document 6).

### PRIOR ART DOCUMENTS

### Patent Documents

Patent Document 1: WO 2004/097007
Patent Document 2: Japanese Patent Application Laid-Open (kokai) No. 2007-109449
Patent Document 3: Japanese Patent Application Laid-Open (kokai) No. 2006-282617
Patent Document 4: Japanese Patent Application Laid-Open (kokai) No. 2007-302607
Patent Document 5: Japanese Patent Application Laid-Open (kokai) No. 2005-23021
Patent Document 6: Japanese Patent Application Laid-Open (kokai) No. 2004-83432

### SUMMARY OF THE INVENTION

### Problems to be Solved by the Invention

However, some of the aforementioned various elastase activity inhibitors exhibit safety and stability which are not sufficiently proven, or provide unsatisfactory elastase activity inhibitory effect. Thus, there is demand for a more effective elastase activity inhibitor.

Under such circumstances, an object of the present invention is to provide an elastase activity inhibitor, an elastase activity inhibiting topical agent, and an elastase activity inhibiting food and beverage composition which have high safety and exhibit satisfactory elastase activity inhibitory effect.

### Means for Solving the Problems

In order to attain the aforementioned object, the present inventors have conducted extensive studies, and have found that Basidiomycetes-X FERM BP-10011 has high safety; can be processed into a form for easy oral ingestion; and exerts elastase activity inhibitory effect. The present invention has been accomplished on the basis of this finding.

In a first mode of the present invention to attain the aforementioned object, there is provided an elastase activity inhibitor characterized by comprising, as an active ingredient, a Basidiomycetes-X FERM BP-10011 dry powder or an extract composition thereof.

A second mode of the present invention is a specific embodiment of the elastase activity inhibitor of the first mode, which is in the form selected from among powder, granule, tablet, capsule, solution, and gel.

In a third mode of the present invention, there is provided an elastase activity inhibiting topical agent characterized by comprising, as an active ingredient, a Basidiomycetes-X FERM BP-10011 dry powder or an extract composition thereof.

A fourth mode of the present invention is a specific embodiment of the elastase activity inhibiting topical agent of the third mode, which is a cosmetic for skin aging inhibition (hereinafter referred to as a “skin anti-aging cosmetic”).

A fifth mode of the present invention is a specific embodiment of the elastase activity inhibiting topical agent of the third mode, which is a hair growing topical agent.

In a sixth mode of the present invention, there is provided an elastase activity inhibiting food and beverage composition characterized by comprising, as an active ingredient, a Basidiomycetes-X FERM BP-10011 dry powder or an extract composition thereof.

A seventh mode of the present invention is a specific embodiment of the elastase activity inhibiting food and beverage composition of the sixth mode, which is a supplement or a drink for skin aging inhibition.

An eighth mode of the present invention is a specific embodiment of the elastase activity inhibiting food and beverage composition of the sixth mode, which is a supplement or a drink for hair growth.

### Effects of the Invention

The present invention employs Basidiomycetes-X, which is highly safe and easy to be taken perorally, to thereby provide an elastase activity inhibitor, an elastase activity inhibiting topical agent, and an elastase activity inhibiting food and beverage composition.

### BRIEF DESCRIPTION OF THE DRAWINGS

FIG. 1 A graph showing a change in relative elastase activity in Test Example 1.

FIG. 2 A photograph of mice of a first hair growth test group (control) in Test Example 2.

FIG. 3 A photograph of mice of a second hair growth test group in Test Example 2.

FIG. 4 A photograph of mice of a third hair growth test group in Test Example 2.

FIG. 5 A photograph of mice of a fourth hair growth test group in Test Example 2.

FIG. 6 A graph showing the hair growth test results conducted in Test Example 2.

### MODES FOR CARRYING OUT THE INVENTION

The elastase activity inhibitor of the present invention contains, as an active ingredient, a Basidiomycetes-X dry powder or an extract composition thereof.

As used herein, the term “Basidiomycetes” refers to a basidiomycete characterized by having beaklike protrusions (i.e., clamps) but no basidium-formability, differing from other basidiomycetes. That is, even when the basidiomycete of the present invention is cultured, only sclerotia (hypha masses) are formed, but the basidia are not formed. Such a basidiomycete was obtained through retrieving a fungus from the natural world. The basidiomycete is isolated and deposited as “Basidiomycetes-X” to the NITE International Patent Organism Depositary (NITE-IPOD) of the National Institute of Technology and Evaluation (NITE) (Accession Number: FERM BP-10011).

The Basidiomycetes-X forms no conidia, or has no asexual generation. For example, when the Basidiomycetes-X is cultured in a potato glucose agar medium, the hyphae (or mycelia) formed through culturing are smooth and have clamps, but form no conidium or fruit body. Through observation of the morphology and color tone of the colony surface, light pinkish hypha masses are formed. In the case where a plurality of hypha masses are formed in a colony concentrically grown from the inoculation site, the hypha masses are interconnected via mycelial strands. Notably, the backside of the colony assumes light pink. When the Basidiomycetes-X is cultured in a glucose-dry yeast agar medium, the hyphae formed through culturing are smooth and have clamps, but form no conidium or fruit body. Through observation of the morphology and color tone of the colony surface, “light pink to white” hypha masses are formed. Hypha masses having a thickness of 5 mm to 6 mm are formed to surround the inoculation site. Notably, the backside of the colony assumes “light pink to white.”

The optimum growth conditions for Basidiomycetes-X include, for example, a pH of 5.0 to 6.0 and a growth temperature of 22° C. to 26° C. The growth allowable conditions include, for example, a pH of 4.0 to 7.5 and a growth temperature of 5° C. to 30° C.

No particular limitation is imposed on the method of culturing the Basidiomycetes-X, and the aforementioned customary method may be employed. In one exemplary mode of culturing, cultured Basidiomycetes-X cells or seed Basidiomycetes-X cells are aseptically inoculated to an agar medium, a sawdust medium, a liquid medium, or the like to which appropriate nutrient sources have been added and which has been sterilized. Culturing is performed at a suitable temperature, whereby hypha masses of the Basidiomycetes-X can be yielded. Notably, the Basidiomycetes-X forms various hypha masses depending on the culture circumstances.

If needed, the thus-formed hypha masses of Basidiomycetes-X are dried, and the dry product is pulverized, to thereby yield a Basidiomycetes-X dry powder, which is an embodiment of the elastase activity inhibitor of the present invention. Alternatively, the dry powder may be formed into an elastase activity inhibitor having a shape of granule, tablet, capsule, solution, gel, etc.

Yet alternatively, a Basidiomycetes-X extract composition may be provided as an active ingredient of the elastase activity inhibitor of the present invention. No particular limitation is imposed on the method of extracting an active ingredient from Basidiomycetes-X hypha masses. In one mode of efficiently extracting cell contents from Basidiomycetes-X hypha masses, preferably, cell walls of the Basidiomycetes-X hypha masses are optionally broken through, for example, freezing in accordance with need. The product is thawed and broken by means of a mixer or the like, and an extract (i.e., Basidiomycetes-X extract composition) is yielded.

No particular limitation is also imposed on the solvent for use in extraction, and water, a lower alcohol, etc. may be used. Also, an extraction solvent further containing an acid, an alkali, or another additive may be used. Extraction is performed at ambient temperature or under heating or pressure. In one customary mode of extraction, Basidiomycetes-X hypha masses are boiled in water for extraction. In an alternative mode, a broken product of Basidiomycetes-X hypha masses is mixed with water or an aqueous mixture containing an alcohol or an alkali, and the resultant mixture is pressurized at, for example, about 100 MPa to about 700 MPa, preferably about 300 MPa to about 600 MPa, for extraction.

An example of the extraction method will next be described. Firstly, frozen Basidiomycetes-X hypha masses are thawed at ambient temperature and broken by means of a mixer. The ratio in amount of the broken Basidiomycetes-X hypha mass to that of water (extraction solvent) is adjusted to, for example, about 1:5. Specifically, the broken Basidiomycetes-X hypha masses (50 g) are placed in a glass bottle, and water (250 mL) is added to the bottle. The bottle is closed. Separately, a towel is placed on the bottom of a pan, and water is poured onto the towel. The glass bottle accommodating the broken product of the Basidiomycetes-X hypha masses is mounted on the towel, and the pan is heated to boil water. Heating is continued for 90 minutes after the start of boiling, and the contents of the glass bottle are cooled. Through solid-liquid phase separation, an extract (Basidiomycetes-X extract composition) and a residue (Basidiomycetes-X extraction residue) are yielded. The pH of the extract is, for example, 6.3 to 6.5. Instead of a broken product of Basidiomycetes-X hypha masses, a Basidiomycetes-X dry powder may be used. In this case, the Basidiomycetes-X dry powder is statically cultured in an aqueous medium for 4 hours to 6 hours, while the solid-liquid medium is suitably stirred. The product is subjected to solid-liquid phase separation, to thereby yield an extract and a residue (Basidiomycetes-X extraction residue).

The thus-obtained extract is optionally concentrated, to thereby provide a Basidiomycetes-X extract composition. No particular limitation is imposed on the extract concentration method, and one exemplary mode is as follows.

Firstly, the obtained extract is transferred to a beaker and is concentrated through heating and evaporation. In the course of concentration, the color of the extract changes from light beige to brown, and vigorous effervescence starts. Evaporation/concentration is further performed. When the extract assumes a tar-like liquid having a pH of 4.9 and a density of 1.25 g/cm³, concentration is stopped. The thus-concentrated extract gives off a soy source-like flavor. At this timing, the average yield of the concentrated extract from the Basidiomycetes-X hypha masses is 12%. Since the viscosity of the thus-obtained concentrated extract steeply increases during cooling of the extract, the extract must be transferred to a storage container immediately after termination of concentration. After cooling, the concentrated extract placed in the storage container is preferably stored in a frozen state.

The thus-obtained Basidiomycetes-X extract composition is optionally dried and processed into a form of powder, granule, tablet, capsule, solution, gel, etc., to thereby provide the elastase activity inhibitor of the present invention. Alternatively, the elastase activity inhibitor of the present invention may be a Basidiomycetes-X dry powder. No particular limitation is imposed on the amount of the elastase activity inhibitor in each of the above products, and the amount may be suitably tuned.

The elastase activity inhibitor of the present invention may be transformed into an elastase activity inhibiting cosmetic and an elastase activity inhibiting food and beverage composition, having any form selected from among powder, granule, tablet, capsule, solution, gel, etc. Through further optional processing of the food and beverage composition, a supplement, a beverage, and the like may be provided. No particular limitation is imposed on the amount of the Basidiomycetes-X dry powder or the Basidiomycetes-X extract composition in each of the elastase activity inhibiting topical agent and the elastase activity inhibiting food and beverage composition, and the amount may be suitably tuned.

As shown in the below-described Examples, the elastase activity inhibitor of the present invention can suppress elastase activity. Thus, the elastase activity inhibitor of the present invention may be formed into a topical agent or an oral (internal) agent. In the preset invention, an embodiment of the topical form is an elastase activity inhibiting topical agent, and examples thereof include a topical drug, a topical quasi-drug, and a cosmetic. Through application thereof to the skin or the scalp, elastase activity can be suppressed. An embodiment of the oral (internal) form is an elastase activity inhibiting food and beverage composition, and examples thereof include an oral drug, an oral quasi-drug, a supplement, a drink, and a beverage.

Typical examples of the elastase activity inhibiting topical agent include a skin anti-aging cosmetic and a hair growing topical agent.

Examples of the skin anti-aging cosmetic include W/O or O/W emulsion cosmetics, cream, lotion, gel, foam, essence, foundation, pack, face wash, and powder. The cosmetic may be a toiletry product such as shampoo, rinse, soap, or body shampoo, and bath preparations. An example of the hair growing topical agent is a hair growth agent which is applied directly to a target site (e.g., the scalp).

The elastase activity inhibiting topical agent may be formulated into a cosmetic, a topical drug, or a quasi-drug by mixing with various ingredients generally used in pharmaceutical preparation (e.g., ingredients generally employed as cosmetic ingredients). Examples of such cosmetic ingredients include an oil, a surfactant, purified water, an alcohol, a chelating agent, a pH adjuster, an antiseptic, a thickener, an emulsifying agent, an emulsion stabilizer, a colorant, and a perfume. There may also be used additional ingredients such as a UV absorber, a whitening agent, a humectant, a sebum secretion inhibitor, a softening agent, a keratin protective agent, a pharmaceutically effective agent, an antioxidant, and a solvent. Any of these ingredients may be used in combination.

Examples of the elastase activity inhibiting food and beverage composition include supplements (e.g., tablets, powders, and capsules), beverages (e.g., liquids and jelly products), and health foods.

As described above, the elastase activity inhibitor of the present invention may be used as an elastase activity inhibiting topical agent or an elastase activity inhibiting food and beverage composition through topical application, taking by mouth, or ingestion, for inhibiting elastase activity. More specifically, the elastase activity inhibitor of the present invention may be used for skin anti-aging (e.g., anti-wrinkling or anti-skin flabbiness) or hair growth. In such an elastase activity inhibiting method or a method for skin anti-aging (e.g., anti-wrinkling or anti-skin flabbiness), no particular limitation is imposed on the method of administering the elastase activity inhibitor or a related composition, and the inhibitor or composition may be administered via topical application, taking by mouth, or ingestion, at an effective amount determined in accordance with a target symptom or the like attributable to elastase activity.

In the present embodiment, taking-by-mouth (oral administration) is preferred, from the viewpoint of ease of ingestion in daily life. In one mode of ingestion, a dry powder of the Basidiomycetes-X extract composition is formulated into tablets of 200 mg to 300 mg, and the tablet is administered once to thrice per day. Preferably the tablet is perorally administered three times a day. Alternatively, a topical agent containing the Basidiomycetes-X extract composition is administered once to twice per day, in an elastase activity inhibition method, a skin anti-aging (e.g., anti-wrinkling or anti-skin flabbiness) method, or a hair growth method. No particular limitation is imposed on the period of time of administration or ingestion, but the time is preferably long. For example, a period of 8 weeks or longer is preferred, with a period of 16 weeks or longer being particularly preferred.

### EXAMPLES

The present invention will next be described in more detail by way of the Examples and the Production Examples of Basidiomycetes-X dry powder and Basidiomycetes-X extract composition.

### Production Example 1

<Separation from Hypha Masses>

### (1) Preparation of Culture Medium

A PSA medium and a PDA medium having the compositions shown in Table 1 were prepared. Each medium was dispensed into a test tube or an Erlenmeyer flask, which was stoppered with Silicosen (registered trademark) (or a cotton plug). These media were sterilized with high-pressure steam at 121° C. for 20 minutes in an autoclave. In the case of a test tube, a hot medium after sterilization was formed into a slant medium, whereas in the case of an Erlenmeyer flask, a sterilized medium was allowed to stand to form a plate medium.

TABLE 1

| PSA medium | PDA medium |
|---|---|
| Petero 200 g (20 min- | Petero 200 g (20 min- |
| boil/extract) | boil/extract) |
| Sucrose 20 g | Glucose 20 g |
| Agar 15 g | Agar 15 g |
| Total volume 1 L | Total volume 1 L |

(2) Separation from Hypha Masses

Larger Basidiomycetes-X hypha masses were broken manually, and slices were cut from Basidiomycetes-X sections with a scalpel which had been flame-sterilized and cooled. The PSA medium and the PDA slant medium of (1) were each inoculated with the Basidiomycetes-X slices by means of tweezers which had been flame-sterilized and cooled. This procedure was performed under aseptic conditions in an aseptic box or a clean bench.

### (3) Culturing in Agar Medium for Production of Hypha Masses

Potato dices (1 cm×1 cm×1 cm) (200 g) were boiled in purified water for 20 minutes and then cooled. The broth was separated from the solid. To a mixture of the broth (potato extract), sucrose (20 g), and agar (1 g, 0.1%), distilled water was added, so that the total volume was adjusted to 1 L, to thereby prepare an agar medium. Although a conventional agar medium has an agar concentration of 1.5 to 2.0 (i.e., 15 g to 20 g based on 1 L of the medium), the agar concentration of this medium was adjusted 0.1%, for facilitating isolation of cultured hypha masses from the agar medium and maintaining the physical strength of Basidiomycetes-X slices, which readily cause sedimentation in a liquid culture medium. The 0.1% agar medium (each 5 mL) was dispensed into test tubes, which were stoppered with Silicosen (registered trademark). These media were sterilized with high-pressure steam at 121° C. for 20 minutes in an autoclave. Thereafter, a slice was cut from the Basidiomycetes-X hypha masses in culturing on the slant medium of Production Example 1. This operation was performed in an aseptic box after completion of aseptic treatment. The slice was inoculated to the 0.1% agar medium under aseptic conditions. The inoculum was cultured in an incubator at 24° C., and was found to generate the organism in 24 to 48 hours. After generation of the organism, culture was continued at 24° C. As a result, hyphae grew on the agar media in 14 days.

### Production Example 2

### <Culturing in Sawdust Medium for Production of Hypha Mass>

### (1) Culturing of Seed Fungus

Water was added to sawdust (1 L), defatted bran (15 g), wheat bran (15 g), and SANPEARL (hypha activator, product of Nippon Paper Industries) (5 g), and the mixture was vigorously stirred. This mixture for culture was adjusted such that when it was firmly gripped, water was exuded (water content of the mixture: about 70%), whereby a sawdust medium was prepared. This culture medium was placed in an Erlenmeyer flask, which was stoppered with Silicosen (registered trademark). Then, the Erlenmeyer flask was subjected to high pressure steam sterilization in an autoclave for 40 minutes at 121° C. Twenty-four hours after sterilization, Basidiomycetes-X hyphae during culture on the slant media in Production Example 1 were inoculated into the sawdust medium within an aseptic box through an aseptic operation. The inoculation was carried out such that no damage was caused to the hyphae, with a sterilized triangular knife being used to cut off a part of the slant medium. The density of the inoculation was adjusted to 20% to 30% of the surface area of the sawdust medium. When the inoculum was cultured at 24° C., the organism was generated in 3 days (in 5 days at the latest). After a lapse of 30 days, the sawdust medium in the Erlenmeyer flask was full of hyphae.

### (2) Generation of Hypha Mass

A sawdust medium was prepared in the same manner as employed in (1). This culture medium was placed in a polypropylene bottle, which was stoppered, and subjected to high pressure steam sterilization in an autoclave for 40 minutes at 121° C. Twenty-four hours after the sterilization, the seed organism cultured in (1) was inoculated into the sawdust medium placed in the polypropylene bottle through an aseptic operation within an aseptic box after aseptic treatment. The density of the inoculation was adjusted such that the surface area of the sawdust medium was virtually covered with the inoculum. When the inoculum was cultured at 24° C., the organism was generated in 48 hours. After a lapse of 60 days, the entire sawdust medium within the polypropylene bottle was full of hyphae. After a further lapse of 40 to 50 days, hyphae spread on the inner wall of the polypropylene bottle, forming mycelial strands. When culture was continued further, hypha masses were formed.

### Production Example 3

### <Production of Basidiomycetes-X Dry Powder>

In order to cause damage to the cell walls of the hyphae and facilitate the leaching-out of the cell contents, fresh Basidiomycetes-X hypha masses obtained in Production Example 2 were frozen. The thus-frozen Basidiomycetes-X hypha masses were thawed at ambient temperature, and crushed by means of a mixer. The product is dried to form a powder (hereinafter referred to as “Basidiomycetes-X dry powder”).

### Production Example 4

### <Production of Basidiomycetes-X Extract Composition Dry Powder>

The Basidiomycetes-X dry powder obtained in Production Example 3 was weighed for 4 kg (dry weight). Water (20 L) was added to the dry powder, and the mixture was subjected to static culturing for 4 to 6 hours under appropriate stirring. Subsequently, the solid contents (hereinafter referred to as “Basidiomycetes-X extraction residue”) in the culture were removed through vacuum filtration, to thereby yield 17.6 kg of a Basidiomycetes-X extract composition (solid content: 8.0%). Then, the product was preliminarily frozen at −40° C. and lyophilized (hereinafter the product being referred to as “Basidiomycetes-X extract composition dry powder”).

### Production Example 5

### <Production of Concentrated Basidiomycetes-X Extract Composition by Boiling>

In order to cause damage to the cell walls of the hyphae and facilitate the leaching-out of the cell contents, fresh Basidiomycetes-X hypha masses were frozen. The thus-frozen Basidiomycetes-X hypha masses were thawed at ambient temperature, and crushed by means of a mixer. Subsequently, the broken Basidiomycetes-X hypha masses (50 g) were placed in a glass bottle, and water (250 mL) was added to the bottle. Then, the bottle was closed. Separately, a towel was placed on the bottom of a pan, and water was poured onto the towel. The glass bottle accommodating the broken product of the Basidiomycetes-X hypha masses was mounted on the towel, and the pan was heated to boil water. Heating was continued for 90 minutes after the start of boiling, and the contents of the glass bottle were cooled. Through solid-liquid phase separation, a Basidiomycetes-X extract composition was yielded. Notably, the pH of the extract was 6.3 to 6.5.

The thus-obtained Basidiomycetes-X extract composition was transferred to a beaker and was concentrated through heating and evaporation. In the course of concentration, the color of the extract composition changed from light beige to brown, and vigorous effervescence started. However, evaporation/concentration was continued further. When the extract assumed a tar-like liquid having a pH of 4.9 and a density of 1.25 g/cm³, concentration was stopped. The thus-concentrated Basidiomycetes-X extract composition gave off a soy source-like flavor. At this timing, the average yield of the concentrated Basidiomycetes-X extract composition from the Basidiomycetes-X hypha masses was 12%. Since the viscosity of the thus-obtained concentrated Basidiomycetes-X extract composition considerably increases during cooling of the extract, the composition was transferred to a storage container simultaneously with termination of concentration. Then, the concentrated composition was cooled and stored in the storage container in a frozen state.

### Production Example 6

### <Production of Basidiomycetes-X Extract Composition by Boiling>

In order to cause damage to the cell walls of the hyphae and facilitate the leaching-out of the cell contents, fresh Basidiomycetes-X hypha masses were frozen. The thus-frozen Basidiomycetes-X hypha masses were thawed at ambient temperature.

The thus-thawed Basidiomycetes-X hypha masses were weighed out (20 g in wet state). The weighed matter was diced into pieces (0.5 cm×0.5 cm×0.5 cm) and transferred into a beaker. Water (100 mL) was added to the beaker, and the mixture was gently boiled at 90° C. until the volume of the mixture was reduced to a volume ½ the initial volume. Subsequently, water was added to the concentrated mixture so as to adjust the volume to the initial value, and the mixture was filtered through gauze, to thereby remove the solid matter. Finally, the filtrate was preliminarily frozen at −40° C. and freeze-dried. Hereinafter, the product may be referred to as a “dry powder of Basidiomycetes-X hot water-extracted composition.” Thereafter, the dry powder was sealed in a container and stored in a refrigerator. The product was employed as a Basidiomycetes-X extract composition of Production Example 6.

### Test Example 1

The dry powder of Basidiomycetes-X hot water-extracted composition produced in Production Example 6 was employed as a test substance. The dry powder was diluted with 50% aqueous dimethylsulfoxide (DMSO), to thereby prepare test liquids 1 to 5 having five different concentrations (0.3, 0.6, 1.2, 2.4, and 4 mg/mL) upon use. The effect of each test liquid on elastase activity was assessed with N-succinyl-Ala-Ala-Ala-p-nitroanilide as a substrate. The specific procedure is as follows.

Firstly, each of test liquids 1 to 5 (50 μL) or a control (50 μL), a 1.25-g/mL solution (50 μL) of an elastase (CAS No. 39445-21-1, Sigma-Aldrich, USA), and a solution (100 L) of N-succinyl-Ala-Ala-Ala-p-nitroanilide (CAS No. 5229914-6, Sigma-Aldrich, USA) were added to a 1.5-mL microtube. The final test substance concentration was adjusted to 75, 150, 300, 600, and 1,000 μg/mL by adding 50% aqueous DMSO, to thereby prepare reaction mixtures. Ultrapure water was used as the control. The test was carried out at n=3.

Each reaction mixture was shaken for 30 seconds by means of a vortex mixer and then incubated at 37° C. for 15 minutes. As a blank, 0.05M Tris-HCl buffer was used instead of elastase. Subsequently, the 1.5-mL microtube was shaken for 10 seconds by means of a vortex mixer and centrifuged at 12,000×g for 2 minutes at room temperature.

Percent elastase activity and percent elastase activity inhibition were calculated from OD₅₁₇ of each test substance and the control by the following equations.

Percent active oxygen retention (%)=(S−SB)/(C−CB)×100

Percent active oxygen deletion (%)={(C−CB)−(S−SB)}/(C-CB)×100

C: OD₅₁₇ of control

CB: Blank OD₅₁₇ of control

S: OD₅₁₇ of test substance

SB: Blank OD₅₁₇ of test substance

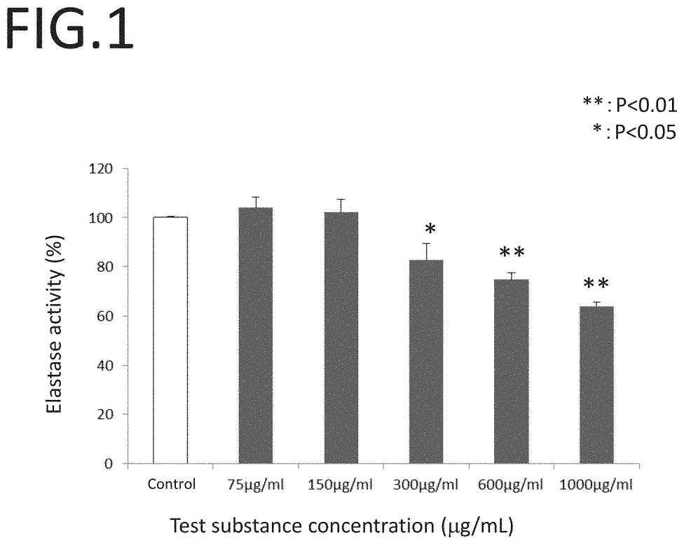

Table 2 shows the percent elastase activity and percent elastase activity inhibition of each test substance (average and standard deviation) with the percent elastase activity of the control being defined as 100%. FIG. 1 is a bar graph of percent elastase activity values. In the case where a significant difference based on the control was found in a group through statistic calculation, the group is marked with an asterisk (*)

TABLE 2

| | Concn. | Percent elastase activity (%) | | | | | |
|---|---|---|---|---|---|---|---|
| | μg/mL | 1 | 2 | 3 | av. | ± | SD |
| Control | — | 100.3 | 100.3 | 99.4 | 100 | ± | 0.5 |
| Test | 75 | 99.9 | 108.6 | 103.3 | 103.9 | ± | 4.4 |
| substance | 150 | 95.9 | 106.0 | 104.2 | 102.0 | ± | 5.4 |
| | 300 | 75.4 | 84.2 | 88.5 | 82.7 | ± | 6.7 |
| | 600 | 71.5 | 76.7 | 75.9 | 74.7 | ± | 2.8 |
| | 1000 | 66.2 | 62.5 | 63.2 | 64.0 | ± | 1.6 |

As is clear from Table 2 and FIG. 1, a significant increase in percent elastase activity inhibition (vs. control) was found in the case where dry powder of Basidiomycetes-X hot-water-extracted composition was used.

### Test Example 2: Hair Growth Test

The dry powder of Basidiomycetes-X hot-water-extracted composition produced in Production Example 6 was formulated into 1% aqueous solution, 3% aqueous solution, and 0.3% aqueous solution, and these solutions were employed as test substances in the following hair growth test.

In the hair growth test, female C57/BL mice (7 weeks old) were employed after back shaving. The mice were divided into four groups including 7 to 8 mice.

A first group was a control group. In a second group, the 1% aqueous solution was applied to the mice once a day. In a third group, the 3% aqueous solution was applied to the mice once a day. In a fourth group, the mice were provided with free taking of the 0.3% aqueous solution.

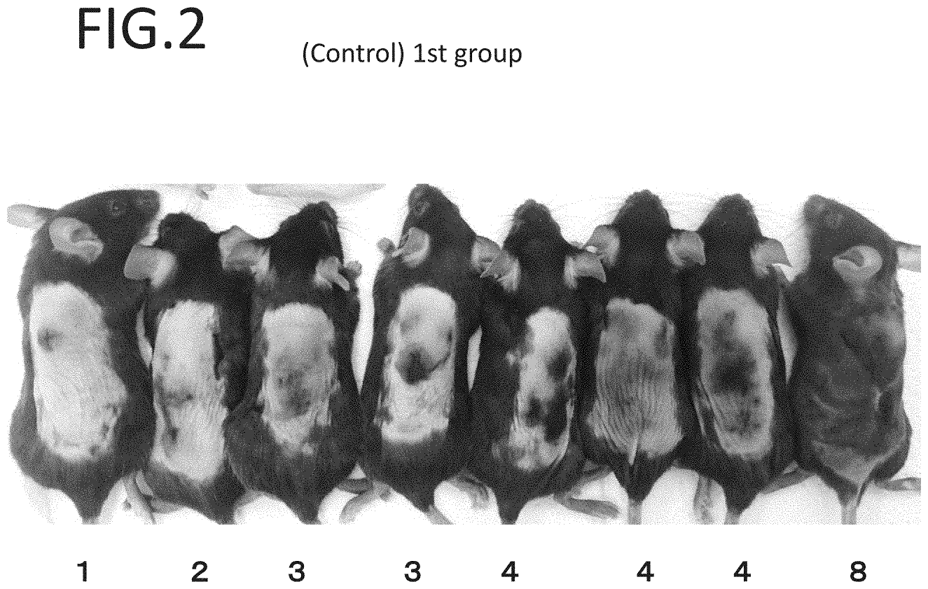

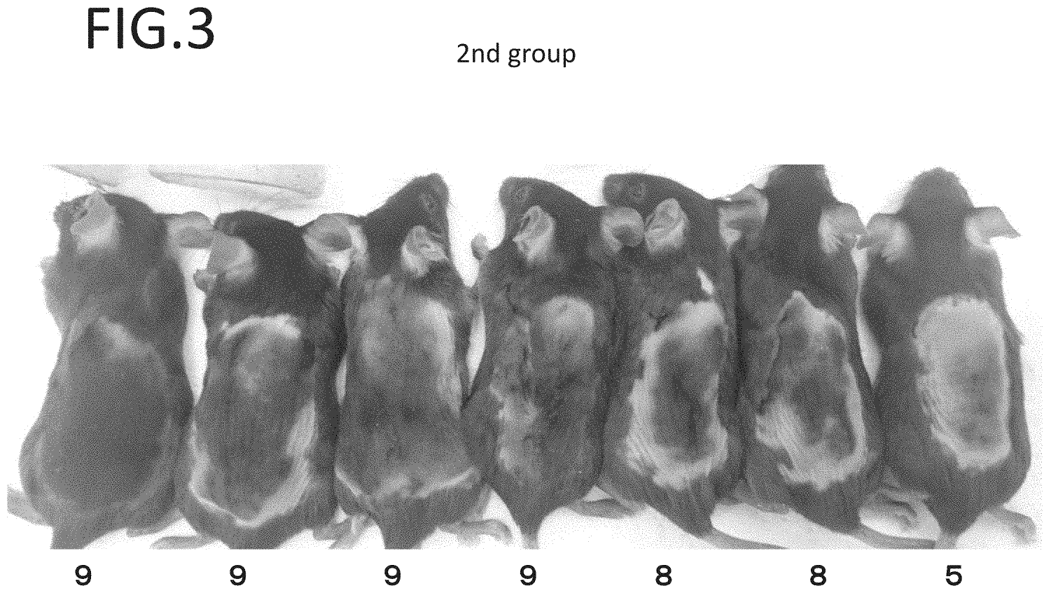

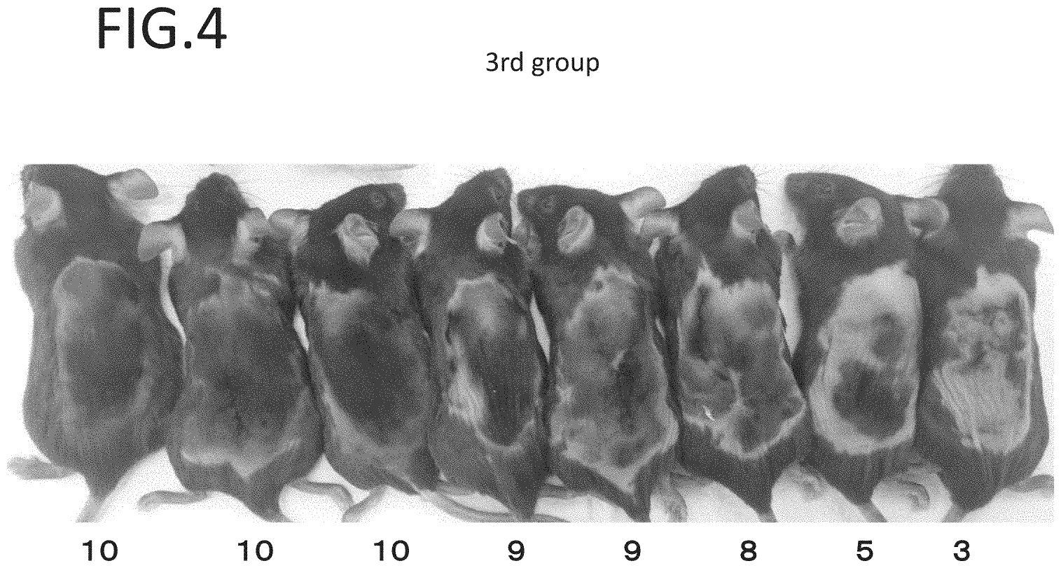

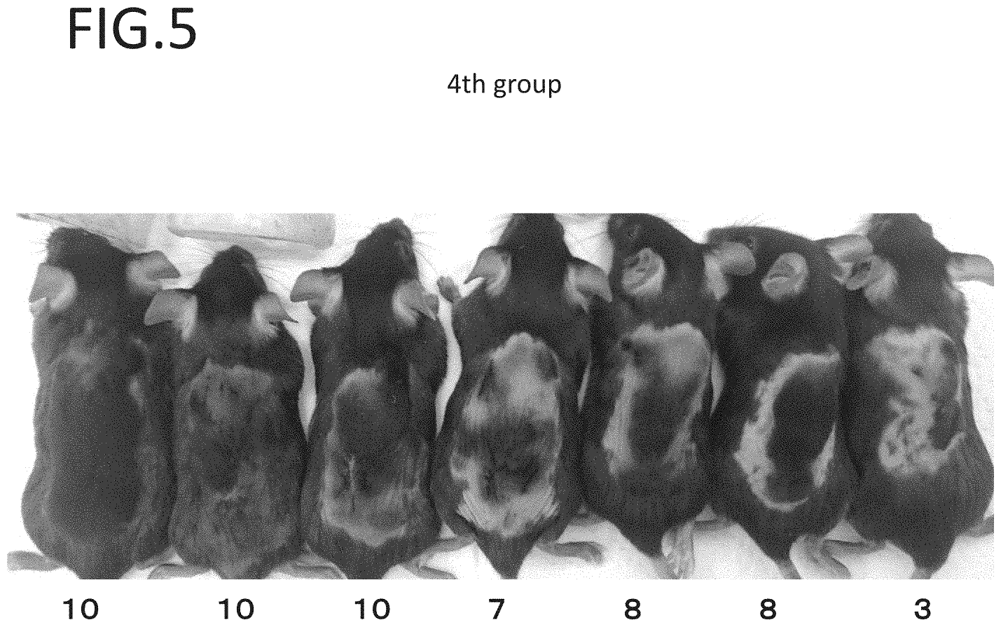

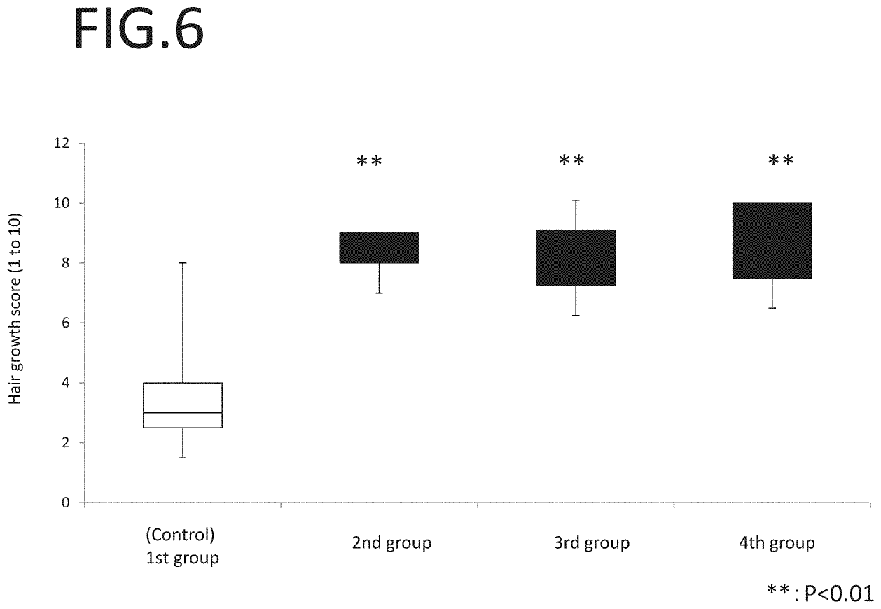

FIGS. 2 to 5 are photographs of the mice taken on day 12 of the hair growth test. FIG. 6 is a graph showing hair growth scores determined to the mice and represented by numerical values 1 to 10 in response to the degree of hair growth.

As is clear from the figures and table, a significant hair growth effect as compared with the control was observed in the second to the fourth groups. In the case where a significant difference based on the control was found in a group through statistic calculation, the group is marked with an asterisk (*)

Accession number

### Basidiomycetes-X FERM BP-10011

### REFERENCES TO DEPOSITED MICROORGANISMS

Name of depositary institution: the International Patent Organism Depositary of the National Institute of Technology and Evaluation
Address of depositary institution: room 120, 2-5-8 Kazusakamatari, Kisarazu, Chiba, Japan
Date of deposition to depositary institution: Feb. 27, 2003
Accession number given to the deposit by the depositary institution: FERM BP-1001

1. An elastase activity inhibitor characterized by comprising, as an active ingredient, a Basidiomycetes-X FERM BP-10011 dry powder or an extract composition thereof.

2. An elastase activity inhibitor according to claim 1, which is in the form selected from among powder, granule, tablet, capsule, solution, and gel.

3. An elastase activity inhibiting topical agent characterized by comprising, as an active ingredient, a Basidiomycetes-X FERM BP-10011 dry powder or an extract composition thereof.

4. An elastase activity inhibiting topical agent according to claim 3, which is a skin anti-aging cosmetic.

5. An elastase activity inhibiting topical agent according to claim 3, which is a hair growing topical agent.

6. An elastase activity inhibiting food and beverage composition characterized by comprising, as an active ingredient, a Basidiomycetes-X FERM BP-10011 dry powder or an extract composition thereof.

7. An elastase activity inhibiting food and beverage composition according to claim 6, which is a supplement or a drink for skin aging inhibition.

8. An elastase activity inhibiting food and beverage composition according to claim 6, which is a supplement or a drink for hair growth.
